SEQUENCE LISTING (19 sequences)

SEQ ID NO: 1 - protein, 8 aa, Artificial
  RUNX3 binding site
PYGPYGGT

SEQ ID NO: 2 - DNA, 19 bp, Artificial
  RUNX3 forward primer
gacagccccaacttcctct

SEQ ID NO: 3 - DNA, 20 bp, Artificial
  RUNX3 reverse primer
cacagtcaccaccgtaccat

SEQ ID NO: 4 - DNA, 20 bp, Artificial
  RUNX3 FRET probe
aaggtggtggcattggggga

SEQ ID NO: 5 - DNA, 20 bp, Artificial
  GAPDH forward primer
gggtgtgaaccatgagaagt

SEQ ID NO: 6 - DNA, 19 bp, Artificial
  GAPDH reverse primer
gactgtggtcatgagtcct

SEQ ID NO: 7 - DNA, 24 bp, Artificial
  GAPDH FRET probe
cagcaatgcctcctgcaccaccaa

SEQ ID NO: 8 - DNA, 20 bp, Artificial
  methylation-specific forward primer
aacgttatcgaggtgttcgc

SEQ ID NO: 9 - DNA, 20 bp, Artificial
  methylation-specific reverse primer
gcgaaattaatacccccgaa

SEQ ID NO: 10 - DNA, 23 bp, Artificial
  unmethylation-specific forward primer
gaatgttattgaggtgtttgtga

SEQ ID NO: 11 - DNA, 21 bp, Artificial
  unmethylation-specific reverse primer
cacaaaattaatacccccaaa

SEQ ID NO: 12 - DNA, 22 bp, Artificial
  miR-532-5p sequence
caugccuugaguguaggaccgu

SEQ ID NO: 13 - DNA, 44 bp, Artificial
  Loop RT primer
ctcaactggtgtcgtggagtcggcaattcagttgagacggtcct

SEQ ID NO: 14 - DNA, 18 bp, Artificial
  miR-532-5p forward primer
gctgggcatgccttgagt

SEQ ID NO: 15 - DNA, 20 bp, Artificial
  universal reverse primer
ctcaactggtgtcgtggagt

SEQ ID NO: 16 - DNA, 18 bp, Artificial
  TaqMan probe
ttcagttgagacggtcct

SEQ ID NO: 17 - DNA, 22 bp, Artificial
  miR-532-5p miR-532-5p miRNA sequence (5′-3′)
caugccuugaguguaggaccgu

SEQ ID NO: 18 - DNA, 22 bp, Artificial
  miR-532-5p miR-532-5p miRNA sequence (3′-5′)
ugccaggaugugaguuccguac

SEQ ID NO: 19 - DNA, 21 bp, Artificial
  RUNX3 mRNA sequence
uagguccuagcagaaggcauu

(19) United States
(12) Patent Application Publication
(10) Pub. No.: US 2010/0227319 A1
(43) Pub. Date: Sep. 9, 2010

(54) USE OF RUNX3 AND MIR-532-5P AS CANCER MARKERS AND THERAPEUTIC TARGETS

(75) Inventors: Dave S.B. Hoon, Los Angeles, CA (US); Minoru Kitago, Santa Monica, CA (US)

Correspondence Address: PERKINS COIE LLP, POST OFFICE BOX 1208, SEATTLE, WA, 98111-1208, US

(73) Assignee: John Wayne Cancer Institute, Santa Monica, CA (US)

(21) Appl. No.: 12/487,592

(22) Filed: Jun. 18, 2009

Related U.S. Application Data
(60) Provisional application No. 61/074,108, filed on Jun. 19, 2008.

(51) Int. Cl. C12Q 1/68; G01N 33/574; C12N 5/09

(52) U.S. Cl. 435/6; 435/7.23; 435/325

(57) ABSTRACT

The invention relates to methods for cancer diagnosis, prognosis, and treatment based on the expression or activity levels of RUNX3 and miR-532-5p. Also disclosed is a method of reducing the inhibition of RUNX3 by miR-532-5p with an agent that interferes with the interaction between RUNX3 and miR-532-5p transcripts.

### RELATED APPLICATION

This application claims priority to U.S. Provisional Application Ser. No. 61/074,108, filed on Jun. 19, 2008, the content of which is incorporated herein by reference in its entirety.

### FUNDING

This invention was made with support in part by grants from NIH, NCI Project II P0 CA029605 and CA012582 grants. Therefore, the U.S. government has certain rights.

### FIELD OF THE INVENTION

The present invention relates in general to cancer. More specifically, the invention relates to the use of RUNX3 (Runt-related transcription factor 3) and miR-532-5p as biomarkers and therapeutic targets for cancer diagnosis, prognosis, and treatment.

### BACKGROUND OF THE INVENTION

The prognosis for patients with American Joint Committee on Cancer (AJCC) stage I/II melanoma is excellent, with an average 10-year survival rate of 85% (1). However, as melanoma progresses from localized to metastatic disease, survival drops significantly. The 10-year survival rate for AJCC stage 1V disease is less than 10% (1). A better understanding of the regulating factors contributing to melanoma tumor growth, progression, and metastases is needed.

Three members of the Runt-related (RUNX) family of genes, RUNX1, RUNX2, and RUNX3 transcription factors, are known as developmental regulators important in the inception and progression of a variety of human cancers and experimentally-induced mouse tumors (2-8). RUNX are transcription factors that are known to function as scaffolds and interact with coregulatory factors often involved in tissue differentiation (9). RUNX proteins are located in the nucleus, whereby downregulation of function has been linked to various cancers (9). Studies have also shown RUNX proteins to regulate gene expression by interacting with chromatin remodeling enzymes (10). RUNX3, in particular, has been shown to be involved in gastric tumor progression. In gastric cancer and other cancers, this gene plays a tumor suppressor role. Hypermethylation of RUNX3 promoter region down-regulates its expression (2, 11). RUNX3 resides on chromosome 1p36, a chromosome site with widely associated aberrations, including in cutaneous melanoma (12, 13).

### SUMMARY OF THE INVENTION

The present invention is based, at least in part, upon the unexpected discovery that the expression of RUNX3 is down-regulated by miR-532-5p, the expression of which is up-regulated in melanoma.

Accordingly, in one aspect, the invention features a method of detecting melanoma. The method comprises providing a test biological sample from a subject and determining the RUNX3 gene expression or protein activity level in the test sample. If the RUNX3 gene expression or protein activity level in the test sample is lower than that in a normal sample, the subject is likely to be suffering from melanoma.

The invention also features another method of detecting melanoma. The method comprises providing a first sample containing melanoma cells and determining the RUNX3 gene expression or protein activity level in the first sample. If the RUNX3 gene expression or protein activity level in the first sample is lower than that in a second sample containing melanoma cells, the melanoma in the first sample is likely to be at a more advanced stage than that in the second sample.

The invention further features a method of predicting the outcome of melanoma. The method comprises providing a first sample containing melanoma cells from a first subject and determining the RUNX3 gene expression or protein activity level in the first sample. If the RUNX3 gene expression or protein activity level in the first sample is higher than that in a second sample containing melanoma cells from a second subject, the overall survival of the first subject is likely to be longer than that of the second subject.

In addition, the invention provides a method of detecting cancer. The method comprises providing a test biological sample from a subject and determining the expression level of miR-532-5p in the test sample. If the expression level of miR-532-5p in the test sample is higher than that in a normal sample, the subject is likely to be suffering from cancer. In some embodiments, the expression level of RUNX3 in the test sample is lower than that in the normal sample.

Another method of the invention for detecting cancer comprises providing a first sample containing cancer cells and determining the expression level of miR-532-5p in the first sample. If the expression level of miR-532-5p in the first sample is higher than that in a second sample containing cancer cells, the cancer in the first sample is likely to be at a more advanced stage than that in the second sample. In some embodiments, the expression level of RUNX3 in the first sample is lower than that in the second sample.

Moreover, the invention provides a method of reducing the inhibition of RUNX3 by miR-532-5p. The method comprises providing a cell expressing a RUNX3 gene and an miR-532-5p gene, and contacting the cell with an agent that interferes with the interaction between RUNX3 and miR-532-5p transcripts. The cell may be a cancer cell. The agent may be an anti-miR-532-5p miRNA.

In some embodiments of the invention, the RUNX3 gene expression level is determined at the mRNA or protein level. The cancer may be melanoma, breast cancer, gastric cancer, pancreas cancer, colon cancer, or esophagus cancer. In some embodiments, the cancer is primary; in other embodiments, the cancer is metastatic.

Unless otherwise defined, all technical and scientific terms used herein have the same meaning as commonly understood by one of ordinary skill in the art to which this invention pertains. In case of conflict, the present document, including definitions, will control. The materials, methods, and examples disclosed herein are illustrative only and not intended to be limiting. Other features, objects, and advantages of the invention will be apparent from the description and the accompanying drawings, and from the claims.

### BRIEF DESCRIPTION OF THE FIGURES

FIG. 1. Relative RUNX3 expression in melanoma cell lines (M1-M11) and normal human melanocytes (HeMn). Melanoma cell lines demonstrated significantly lower RUNX3 gene expression than normal melanocyte line HeMn (p<0.001). The assays were run in triplicate.

FIG. 2. The ratio of expression levels of miR-532-5p in melanoma cell lines compared to HeMn by qRT. Expression of miR-532-5p in melanoma lines was higher than in normal melanocytes (HeMn) by qRT. The assays were performed in duplicate.

FIG. 3. Expression level of miR-532-5p in primary and metastatic melanoma tumors. Metastatic melanoma tumors showed significantly higher expression level of miR-532-5p compared to primary melanomas (p=0.0012). The assays were performed in duplicate.

FIG. 4. Expression of RUNX3 mRNA levels in anti-miR-532-5p-transfected melanoma cells compared to negative control transfected cells. Anti-miR-532-5p transfected melanoma cells showed up-regulation of RUNX3 mRNA levels compared to negative control transfected cells. The experiments represent mean of duplicates.

FIG. 5. Expression of RUNX3 protein levels in anti-miR-532-5p-transfected melanoma cells compared to negative control transfected cells by flow cytometry. Anti-miR-532-5p transfected cells (grey) showed upregulation of RUNX3 protein levels compared to negative control transfected cells (black). The studies were performed in duplicate.

### DETAILED DESCRIPTION OF THE INVENTION

The present invention relates to the use of RUNX3 and miR-532-5p as biomarkers and therapeutic targets for cancer diagnosis, prognosis, and treatment.

RUNX3 and miR-532-5p are known in the art. For example, the GenBank Accession Number for a human RUNX3 is NM_{—}004350; the miRBase Entry Number for miR-532-5p is MI0003205.

One object of the invention is to provide methods for diagnosing cancer.

In one method, a test biological sample from a subject is provided. The RUNX3 gene expression or protein activity level in the test sample is determined, e.g., by detecting and quantifying RUNX3 mRNA or protein level, or RUNX3 protein activity level, using a number of means well known in the art. The RUNX3 gene expression or protein activity level in the test sample is compared with the RUNX3 gene expression or protein activity level in a normal sample. If the RUNX3 gene expression or protein activity level in the test sample is lower than the RUNX3 gene expression or protein activity level in a normal sample, the subject is likely to be suffering from melanoma, either primary or metastatic.

In another method, a test biological sample from a subject is provided. The expression level of miR-532-5p in the test sample is determined, e.g., by detecting and quantifying miR-532-5p transcript level using a number of means well known in the art. The expression level of miR-532-5p in the test sample is compared with the expression level of miR-532-5p in a normal sample. If the expression level of miR-532-5p in the test sample is higher than the expression level of miR-532-5p in a normal sample, the subject is likely to be suffering from cancer, either primary or metastatic.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

As used herein, “cancer” refers to a disease or disorder characterized by uncontrolled division of cells and the ability of these cells to spread, either by direct growth into adjacent tissue through invasion, or by implantation into distant sites by metastasis. Exemplary cancers include, but are not limited to, carcinoma, adenoma, lymphoma, leukemia, sarcoma, mesothelioma, glioma, germinoma, choriocarcinoma, prostate cancer, lung cancer, breast cancer, colorectal cancer, gastrointestinal cancer, bladder cancer, pancreatic cancer, endometrial cancer, ovarian cancer, melanoma, brain cancer, testicular cancer, kidney cancer, skin cancer, thyroid cancer, head and neck cancer, liver cancer, esophageal cancer, gastric cancer, intestinal cancer, colon cancer, rectal cancer, myeloma, neuroblastoma, and retinoblastoma. Preferably, the cancer is melanoma, breast cancer, gastric cancer, pancreas cancer, colon cancer, or esophagus cancer.

The test sample may be obtained from tissues where cancer may originate or metastasize. Such tissues are known in the art. For example, it is well known that melanoma may originate from skin, bowel, and eye, and metastasize to stomach, esophagus, bowel, lung, brain, skin, lymph node, breast, and other tissues.

The test sample may also be obtained from body fluids where cancer cells may be present. Such body fluids are also known in the art, including, without limitation, blood, serum, plasma, bone marrow, cerebral spinal fluid, peritoneal/pleural fluid, lymph fluid, ascite, serous fluid, sputum, lacrimal fluid, stool, and urine.

A test sample may be prepared using any of the methods known in the art. The expression level of RUNX3 or miR-532-5p in the test sample may be determined, e.g., by detecting and quantifying RUNX3 mRNA, miR-532-5p RNA, or RUNX3 protein level using a number of means well known in the art.

To measure RNA levels, cells in biological samples can be lysed and the RNA levels in the lysates determined by any of a variety of methods familiar to those in the art. Such methods include, without limitation, hybridization assays using detectably labeled gene-specific DNA or RNA probes and quantitative or semi-quantitative real-time RT-PCR methodologies using appropriate gene-specific oligonucleotide primers. Alternatively, quantitative or semi-quantitative in situ hybridization assays can be carried out using, for example, unlysed tissues or cell suspensions, and detectably (e.g., fluorescently or enzyme-) labeled DNA or RNA probes. Additional methods for quantifying mRNA levels include RNA protection assay (RPA), cDNA and oligonucleotide microarrays, and colorimetric probe based assays.

Methods for measuring protein levels in biological samples are also known in the art. Many such methods employ antibodies (e.g., monoclonal or polyclonal antibodies) that bind specifically to target proteins. In such assays, an antibody itself or a secondary antibody that binds to it can be detectably labeled. Alternatively, the antibody can be conjugated with biotin, and detectably labeled avidin can be used to detect the presence of the biotinylated antibody. Combinations of these approaches (including “multi-layer sandwich” assays) familiar to those in the art can be used to enhance the sensitivity of the methodologies. Some of these protein-measuring assays (e.g., ELISA or Western blot) can be applied to lysates of test cells, and others (e.g., immunohistological methods or fluorescence flow cytometry) applied to unlysed tissues or cell suspensions. Methods of measuring the amount of a label depend on the nature of the label and are known in the art. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

RUNX3 is a transcription factor. It binds to the core DNA sequence 5′-PYGPYGGT-3′ found in a number of enhancers and promoters, and can either activate or suppress transcription. The activity of the RUNX3 protein can be determined using any of the methods known in the art. For example, the protein activity of RUNX3 may be determined by measuring the expression levels of genes regulated by RUNX3, cell proliferation assay, apoptosis assay, or tumorigenesis assay.

As used herein, a “normal sample” is a sample prepared from a normal subject, a normal tissue, or a normal body fluid.

Another object of the invention is to provide methods for determining cancer stages using techniques similar to those described above.

In one method, a first sample containing melanoma cells is provided, and the RUNX3 gene expression or protein activity level in the sample is determined. The RUNX3 gene expression or protein activity level in the first sample is compared to the RUNX3 gene expression or protein activity level in a second sample containing melanoma cells. If the RUNX3 gene expression or protein activity level in the first sample is lower than the RUNX3 gene expression or protein activity level in the second sample, the melanoma in the first sample is likely to be at a more advanced stage than the melanoma in the second sample. If the RUNX3 gene expression or protein activity level in the first sample is higher than the RUNX3 gene expression or protein activity level in the second sample, the melanoma in the first sample is likely to be at a less advanced stage than the melanoma in the second sample.

In another method, a first sample containing cancer cells is provided, and the expression level of RUNX3 in the sample is determined. The expression level of miR-532-5p in the first sample is compared to the expression level of miR-532-5p in a second sample containing cancer cells. If the expression level of miR-532-5p in the first sample is higher than the expression level of miR-532-5p in the second sample, the cancer in the first sample is likely to be at a more advanced stage than the cancer in the second sample. If the expression level of miR-532-5p in the first sample is lower than the expression level of miR-532-5p in the second sample, the cancer in the first sample is likely to be at a less advanced stage than the cancer in the second sample.

This method can be used to compare the stages of cancer in different subjects if the first and second samples are obtained from different subjects. On the other hand, if the first and second samples are obtained from the same subject at different time points (e.g., before and after a cancer treatment), the method can be used to monitor cancer progression or regression and evaluate the effectiveness of the treatment.

The invention further provides methods for predicting the outcome of cancer using techniques similar to those described above.

In one method, a first sample containing melanoma cells from a first subject is provided. The RUNX3 gene expression or protein activity level in this sample is determined and compared with the RUNX3 gene expression or protein activity level in a second sample containing melanoma cells from a second subject. If the RUNX3 gene expression or protein activity level in the first sample is higher than the RUNX3 gene expression or protein activity level in the second sample, the overall survival of the first subject is likely to be longer than the overall survival of the second subject. If the RUNX3 gene expression or protein activity level in the first sample is lower than the RUNX3 gene expression or protein activity level in the second sample, the overall survival of the first subject is likely to be shorter than the overall survival of the second subject.

In another method, a first sample containing cancer cells from a first subject is provided. The expression level of miR-532-5p in this sample is determined and compared with the expression level of miR-532-5p in a second sample containing cancer cells from a second subject. If the expression level of miR-532-5p in the first sample is lower than the expression level of miR-532-5p in the second sample, the overall survival of the first subject is likely to be longer than the overall survival of the second subject. If the expression level of miR-532-5p in the first sample is higher than the expression level of miR-532-5p in the second sample, the overall survival of the first subject is likely to be shorter than the overall survival of the second subject.

This method can be used to compare the overall survival of different subjects if the first and second samples are obtained from different subjects. On the other hand, if the first and second samples are obtained from the same subject at different time points (e.g., the first subject is a subject before a cancer treatment; the second subject is the same subject after the treatment), the method can be used to monitor the overall survival of the subject and evaluate the effectiveness of the treatment.

The discovery of the decreased RUNX3 expression, increased miR-532-5p expression, and the interaction between RUNX3 and miR-532-5p in melanoma is useful for identifying candidate compounds for modulating RUNX3 and miR-532-5p gene expression, protein activity, or transcript interaction in vitro and in vivo and for treating cancer.

In one method of the invention, a system (e.g., a cell such as a melanoma cell) containing a RUNX3 gene or protein is contacted with a test compound. The RUNX3 gene expression or protein activity levels in the system prior to and after the contacting step are compared. If the RUNX3 gene expression or protein activity level increases after the contacting step, the test compound is identified as a candidate for enhancing RUNX3 gene expression or protein activity in a cell and for treating melanoma.

In another method of the invention, a system (e.g., a cell such as a cancer cell) containing an miR-532-5p gene is contacted with a test compound. The expression levels of miR-532-5p in the system prior to and after the contacting step are compared. If the expression level of miR-532-5p decreases after the contacting step, the test compound is identified as a candidate for inhibiting miR-532-5p expression in a cell and for treating cancer.

In still another method of the invention, a system (e.g., a cell such as a cancer cell) containing a RUNX3 gene, or a transcript thereof, and an miR-532-5p gene, or a transcript thereof, is contacted with a test compound. If the compound interferes with the interaction between the RUNX3 and miR-532-5p transcripts, the test compound is identified as a candidate for inhibiting the interaction between RUNX3 and miR-532-5p transcripts in a cell and for treating cancer.

The test compounds can be obtained using any of the numerous approaches (e.g., combinatorial library methods) known in the art. Such libraries include, without limitation, peptide libraries, nucleic acid libraries, peptoid libraries, spatially addressable parallel solid phase or solution phase libraries, synthetic libraries obtained by deconvolution or affinity chromatography selection, and the “one-bead one-compound” libraries. Compounds in the last three libraries can be peptides, non-peptide oligomers, or small molecules. Examples of methods for synthesizing molecular libraries can be found in the art.

The compounds so identified are within the invention. These compounds and other compounds known to enhance RUNX3 gene expression or protein activity, inhibit miR-532-5p expression, or interfere with the interaction between RUNX3 and miR-532-5p transcripts can be used to modulate RUNX3 and miR-532-5p gene expression, protein activity, or transcript interaction in vitro and in vivo.

Accordingly, in one method of the invention, a melanoma cell is contacted with a compound of the invention (e.g., a nucleic acid encoding a RUNX3 gene, a RUNX3 protein, their fragments or functional equivalents, and the like), thereby enhancing RUNX3 gene expression or protein activity in the cell.

In another method of the invention, a cancer cell is contacted with a compound of the invention (e.g., an anti-sense RNA, a ribonuclease, and the like), thereby inhibiting miR532-5p expression.

In still another method of the invention, a cell (e.g., a cancer cell) expressing RUNX3 and miR-532-5p is provided. The cell is contacted with a compound of the invention (e.g., an anti-sense RNA such as anti-miR-532-5p miRNA, a ribonuclease, and the like), thereby interfering with the interaction between RUNX3 and miR-532-5p transcripts in the cell.

A compound of the invention can also be used to treat cancer (e.g., melanoma) by administering an effective amount of the compound to a subject suffering from cancer. In some embodiments, a compound that enhances RUNX3 gene expression or protein activity is administered to a subject suffering from melanoma. In some embodiments, a compound that inhibits miR532-5p expression or interferes with the interaction between RUNX3 and miR-532-5p transcripts is administered to a subject suffering from cancer.

A subject to be treated may be identified in the judgment of the subject or a health care professional, and can be subjective (e.g., opinion) or objective (e.g., measurable by a test or diagnostic method such as those described above).

A “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a compound that is capable of producing a medically desirable result in a treated subject. The medically desirable result may be objective (i.e., measurable by some test or marker) or subjective (i.e., subject gives an indication of or feels an effect).

For treatment of cancer, a compound is preferably delivered directly to tumor cells, e.g., to a tumor or a tumor bed following surgical excision of the tumor, in order to treat any remaining tumor cells.

The compounds of the invention may be incorporated into pharmaceutical compositions. Such compositions typically include the compounds and pharmaceutically acceptable carriers. “Pharmaceutically acceptable carriers” include solvents, dispersion media, coatings, antibacterial and antifungal agents, isotonic and absorption delaying agents, and the like, compatible with pharmaceutical administration.

A pharmaceutical composition is normally formulated to be compatible with its intended route of administration. Examples of routes of administration include parenteral (e.g., intravenous), intradermal, subcutaneous, oral (e.g., inhalation), transdermal (topical), transmucosal, and rectal administration.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are typically in the range of 0.01-100.0 mg/kg. Wide variations in the needed dosage are to be expected in view of the variety of compounds available and the different efficiencies of various routes of administration.

The following example is intended to illustrate, but not to limit, the scope of the invention. While such example is typical of those that might be used, other procedures known to those skilled in the art may alternatively be utilized. Indeed, those of ordinary skill in the art can readily envision and produce further embodiments, based on the teachings herein, without undue experimentation.

### Example

### Regulation of RUNX3 Tumor Suppressor Gene Expression in Cutaneous Melanoma

### Statement of Translational Relevance

In malignant cutaneous melanoma there is limited number of tumor suppressor genes known to be downregulated during tumor metastasis. The study identifies the downregulation of the tumor suppressor gene RUNX3 in cutaneous melanoma during tumor progression. These studies suggest RUNX3 expression level may be a potential target for therapy and diagnosis. Identification of regulatory mechanisms of tumor suppressor genes may allow for the development of new approaches of targeted therapeutics. The mechanism of RUNX3 mRNA downregulation was demonstrated to be through miR 532-5p. This novel finding suggests that blocking miR-532-5p may be a potential approach to upregulate RUNX3 expression as a treatment of cutaneous melanoma. The study demonstrates specific microRNA to a tumor suppressor gene may be a significant epigenetic mechanism in regulating tumor development and progression.

### Abstract

Purpose: RUNX3 is a known tumor-suppressor gene in several carcinomas. Aberration in RUNX3 expression has not been described for cutaneous melanoma. Therefore, we assessed the expression of RUNX3 in cutaneous melanoma and its regulatory mechanisms relative to tumor progression.

Experimental Design: Expression of RUNX3 mRNA and miR-532-5p (microRNA) were assessed in melanoma lines, and primary and metastatic melanoma tumors.

Results: RUNX3 mRNA expression was downregulated in 11 of 11 (100%) metastatic melanoma lines relative to normal melanocytes (p<0.001). Among 123 primary and metastatic melanoma tumors and 12 normal skin samples, RUNX3 expression was downregulated significantly in primary melanomas (n=82; p=0.02) or melanoma metastasis (n=41; p<0.0001) versus normal skin (n=12). This suggested that RUNX3 downregulation may play a role in the development and progression of melanoma. RUNX3 promoter region hypermethylation was assessed as a possible regulator of RUNX3 expression using methylation-specific PCR. Assessment of RUNX3 promoter region methylation showed that only 5 of 17 (29%) melanoma lines, 2 of 52 (4%) primary melanomas, and 5 of 30 (17%) metastatic melanomas had hypermethylation of the promoter region. A microRNA (miR-532-5p) was identified as a target of RUNX3 mRNA sequences. miR-532-5p expression was shown to be significantly upregulated in melanoma lines and metastatic melanoma tumors relative to normal melanocytes and primary melanomas, respectively. To investigate the relation between RUNX3 and miR-532-5p, anti-miR-532-5p was transfected into melanoma lines. Inhibition of miR-532-5p upregulated both RUNX3 mRNA and protein expression.

Conclusions: RUNX3 is downregulated during melanoma progression and miR-532-5p is a regulatory factor of RUNX3 expression.

### Introduction

There have been no major reports of altered RUNX3 expression in cutaneous melanoma. Based upon patterns discerned from other malignancies, we believed that RUNX3 expression in melanoma may be suppressed, and that levels of expression may relate to melanoma progression as in other cancers. We found that RUNX3 expression is downregulated in metastatic melanomas compared to primary tumors. The role of promoter region hypermethylation and microRNA (miRNA) was investigated to examine possible mechanisms for RUNX3 expression downregulation.

### Materials and Methods

### Cell Lines

Eleven melanoma lines (M1-M11) established from metastatic tumors of patients treated at John Wayne Cancer Institute (JWCI)/St. Johns Health Center (SJHC) were maintained in RPMI-1640 medium (Gibco, Carlsbad, Calif.) supplemented with 10% heat-inactivated fetal bovine serum, 1% penicillin, and streptomycin (14). The pancreas cancer cell line COLO 357 (gift from Dr. M. Korc) served as a positive control for RUNX3 expression. Kato III (ATCC, Manassas, Va.), a gastric cancer cell line that expresses RUNX3 in low copy numbers was used as a negative control. HeMn-MP (Cascade Biologics, Portland, Oreg.), a moderately pigmented human melanocyte cell line, was maintained in basal media 254 supplemented with human melanocyte growth supplement. Cell lines were kept in 75 cm³ flasks at 37° C. in a 5% CO₂ incubator.

### Melanoma Specimens

Approval for the use of patient specimens was granted by a joint JWCI/SJHC Institutional Review Board. The JWCI melanoma patient database and SJHC Cancer Registry were used to identify all patients treated for primary or metastatic melanoma between 1995 and 2004. The final pathological diagnosis and availability of all paraffin-embedded (PE) melanomas were confirmed with the SJHC Department of Pathology. Only specimens with ≧60% tumor cells evident during light microscopic analysis were further processed and analyzed. The study of population demographics is given in Table 1.

TABLE 1
Patient Characteristics
Patient Characteristics

| | | | | |
|---|---|---|---|---|
| Patients (n) | Men | | 67 | (54.5%) |
| | Women | | 56 | (45.5%) |
| | | Total | 123 | (100%) |
| Mean Age | | | 65 | (range, 14-90) |
| (yrs) | | | | |
| Median | | | 44 | (range, 3-149) |
| Follow-up | | | | |
| (mos) | | | | |

Tumor Characteristics

| | | | | |
|---|---|---|---|---|
| Tumors | Primary | AJCC stage I | 45 | (54.9%) |
| Assessed (n) | | AJCC stage II | 21 | (25.6%) |
| | | AJCC stage III | 16 | (19.5%) |
| | Total Primary | | 82 | (100%) |
| | Sites | Superficial Spreading | 45 | (54.9%) |
| | | Nodular | 19 | (23.1%) |
| | | Desmoplastic | 8 | (9.8%) |
| | | Lentigo Maligna | 6 | (7.3%) |
| | | Acral Lentiginous | 4 | (4.9%) |
| | | Mean Breslow Depth (mm) | 2.06 | (range, 0.19-11) |
| | | Clark Level II | 14 | (17.1%) |
| | | Clark Level III | 20 | (24.4%) |
| | | Clark Level IV | 32 | (39%) |
| | | Clark Level V | 13 | (15.9%) |
| | | Clark Level Unknown | 3 | (3.6%) |
| | | Ulceration | 14 | (17.1%) |
| | Metastasis | AJCC stage III | 19 | (46%) |
| | | AJCC stage IV | 22 | (54%) |
| | Total | | 41 | (100%) |
| | Metastatic | | | |
| | Sites | Subcutaneous tissue | 8 | (36%) |
| | | Lung | 6 | (27%) |
| | | Brain | 2 | (9%) |
| | | Gastrointestinal | 4 | (18%) |
| | | Distant lymph nodes | 1 | (5%) |
| | | Breast | 1 | (5%) |

A total of 123 melanomas were assayed, including both primary (N=82) and metastatic tumors (N=41). A list of patients with non-malignant nevi, skin, lymph nodes, and visceral tissues were obtained from the database coordinator to serve as normal controls.

miRNA, RNA, and DNA Isolation

Genomic DNA was extracted from cell lines using DNAzol Genomic DNA Isolation Reagent (Molecular Research Center, Inc., Cincinnati, Ohio) according to the manufacturer's recommendations.

Total RNA for the mRNA study was extracted with TRI Reagent (Molecular Research Center, Inc.) according to the manufacturer's protocol. Total RNA for miRNA study was extracted from cells by using mirVana™ miRNA Isolation Kit (Ambion Inc., Austin, Tex.) according to the manufacturer's recommendations. Quality and quantity of extracted DNA and RNA were measured by UV absorption spectrophotometry and RiboGreen (Molecular Probes, Eugene, Oreg.). Only specimens with high-quality mRNA were included in the study.

For RNA extraction from PE tissues, 7 sections of 10 μm thickness were cut from each paraffin block using a new sterile microtome blade for each block. Sections were then deparaffinized and digested with proteinase K prior to RNA extraction using the RNAwiz RNA isolation reagent (Ambion Inc.) following a modification of the manufacturer's protocol (15). Pellet Paint NF (Novagen, Madison, Wis.) was used as a carrier in the RNA precipitation step.

### Quantitative Real-Time PCR Primers and Probes

RUNX3 primers were designed to span at least one exon-intron-exon region to optimally amplify cDNA and minimize genomic DNA amplification. To account for degradation of RNA in PE tissue, primers were designed to amplify cDNA amplicons of ≦150 bp. Amplicon size was confirmed by gel electrophoresis. Primer and probe sequences for RUNX3 and glyceraldehyde-3-phosphate dehydrogenase (GAPDH) are provided below. RUNX3: 5′-GACAGCCCCAACTTCCTCT-3′ (forward), 5′-CACAGTCACCACCGT ACCAT-3′ (reverse), 5′-FAM-AAGGTGGTGGCATTGGGGGA-BHQ-1-3′ (FRET probe); GAPDH: 5′-GGGTGTGAACCATGAGAAGT-3′ (forward), 5′-GACTGTGGTCATGA GTCCT-3′ (reverse), and 5′-FAM-CAGCA ATGCCTCCTGCACCACCAA-BHQ-1-3′ (FRET probe).

Quantitative Real-Time RT-PCR (qRT)

Reverse transcription of total RNA was performed using Moloney murine leukemia virus reverse transcriptase (Promega, Madison, Wis.) as previously described (16). Probe-based qRT was performed in a 96-well plate format using the ABI Prism 7000 (Applied Biosystems Inc., Foster City, Calif.) in a blinded fashion. A standard amount of total RNA (250 ng) was used for all reactions. The qRT assay was optimized using established melanoma cell lines and PE metastatic tumors. The accuracy and reproducibility of the assay was ensured by comparing qRT results from different sections of the same tumor and including the necessary controls for all reactions. We transferred 5 μL of cDNA from 250 ng of total RNA to a well of a 96-well PCR plate in which 0.2 μM of each primer, 0.3 μM FRET probe and iTaq custom Supermix (Bio-Rad Laboratories, Hercules, Calif.) to a final volume of 25 μL. Each PCR reaction was composed of 45 cycles at 95° C. for 60 sec, 60° C. for 60 sec, and 72° C. for 60 sec for RUNX3; and 45 cycles at 95° C. for 60 sec, 55° C. for 60 sec, and 72° C. for 60 sec for GAPDH. Each assay was performed with standard curves, positive controls (cell lines), negative controls (cell lines) and reagent controls (reagents without cDNA) (17). Expression of the housekeeping gene GAPDH was assessed in each sample to verify mRNA integrity. RUNX3 expression was designated as a ratio of RUNX3/GAPDH mRNA units. RUNX3 mRNA expression ratios from established melanoma cell lines were compared to the mean mRNA expression ratio from normal melanocytes. Patient specimens were normalized with respect to the mean RUNX3/GAPDH mRNA expression ratios from normal tissues to account for low background levels of RUNX3 expression in melanoma tissues. All assays were performed in triplicate.

### DNA Extraction and Sodium Bisulfite Modification (SBM)

DNA was extracted from a subset of PE melanoma specimens (total N=82) previously assayed by qRT. Light microscopy was used to confirm tumor location and assess tumor tissue for microdissection. Additional sections from the tumor block were mounted on glass slides and microdissected under light microscopy. Dissected tissues were digested with 50 μL of proteinase K-containing lysis buffer at 50° C. for 5 hr, followed by heat deactivation of proteinase K at 95° C. for 10 min. Sodium bisulfite modification (SBM) was applied on extracted genomic DNA of tissue specimens or cell lines for MSP or bisulfite sequencing as described previously (18).

### Detection of Methylated RUNX3

SBM was applied on extracted genomic DNA of tissue specimens and cell lines for MSP (18). Methylation-specific and unmethylated-specific primer sets were designed; optimization for MSP included annealing temperature, Mg²⁺ concentration, and cycle number for specific amplification of the methylated and unmethylated target sequences. The primers were dye-labeled for automatic detection by capillary array electrophoresis (CAE). The methylation-specific primer set was as follows: forward, 5′-D4-AACGTTATCGAGGTGTTCGC-3′; and reverse, 5′-GCGAAATTAATACCCCCGAA-3′. The unmethylation-specific primer set was as follows: forward, 5′-D3-GAATGTTATTGAGGTGTTTGTGA-3′; and reverse, 5′-CACAAAATTAATACCCCCAAA-3′. PCR amplification was performed in a 10 μL reaction volume with 1 μL template for 36 cycles of 30 sec at 94° C., 30 sec at 63° C. for methylated and 60° C. for unmethylated reaction, and 30 sec at 72° C., followed by a 7-min final extension at 72° C. The PCR reaction mixture consisted of 0.3 μM of each primer, 1 U of AmpliTaq Gold polymerase (Applied Biosystems, Inc.), 200 μM of each deoxynucleoside triphosphate, 2.5 mM MgCl₂, and PCR buffer to a final volume of 10 μL. A universal unmethylated control was synthesized from normal DNA by phi-29 DNA polymerase and served as a positive unmethylated control (19). Unmodified lymphocyte DNA was used as a negative control for methylated and unmethylated reactions. SssI Methylase (New England Bio Labs, Beverly, Mass.) treated lymphocyte DNA was used as a positive methylated control. PCR products were detected and analyzed by CAE (CEQ 8000XL; Beckman Coulter, Inc., Fullerton, Calif.) with CEQ 8000XL software version 8.0 (Beckman Coulter) as described previously (20).

Detection of miRNA by Real-Time Stem-Loop RT-PCR

Reverse transcriptase reactions contained total RNA, 50 nM stem-loop RT primer for miR-532-5p, and TaqMan MicroRNA reverse Transcription kit (1×RT buffer, 0.25 mM each of dNTPs, 3.33 U/μL MultiScribe reverse transcriptase and 0.25 U/μL RNase inhibitor; Applied Biosystems Inc.) following the manufacturer's protocol. The reactions were incubated in a Thermocycler in a 384 well plate for 30 min at 16° C., 30 min at 42° C., 5 min at 85° C., and then held at 4° C. All reverse transcriptase reactions, including no-template controls and RT minus controls, were run in duplicate.

All primers and probes are designed based on miRNA sequences released by the Sanger Institute (21). The primer and probe was designed by Primer Express software (Applied Biosystems, Inc.) as previously described (22, 23). The miR-532-5p sequence is 5′-CAUGCCUUGAGUGUAGGACCGU-3′. The Loop RT primer is 5′-CTCAACTGGTGTCGTGGAGTCGGCAATTCAGTTGAGACGGTCCT-3′. The forward primer is 5′-GCTGGGCATGCCTTGAGT-3′. The universal reverse is 5′-CTCAACTGGTGTCGTGGAGT-3′. The TaqMan Probe is (6-FAM)-TTCAGTTGAGACGGTCCT-MGB. The underlined sequences are specific for miR-532-5p.

qRT was performed in a 384 well plate format using The ABI Prism 7000 (Applied Biosystems, Inc.) in blinded fashion. The 10 μL PCR included 0.67 μL RT product, 1× TaqMan Universal PCR Master Mix (Applied Biosystems, Inc.), 0.2 μM TaqMan probe, 1.5 μM forward primer and 0.7 μM reverse primer. The reactions were incubated in a 384 well plate at 95° C. for 10 min, followed by 40 cycles of 95° C. for 15 sec and 60° C. for 1 min. All reactions were run in triplicate. Standard curves were generated by using a threshold cycle (Ct) of eight serially diluted (10 to 10⁸ copies) plasmids containing stem-loop RT cDNA of miR-532-5p. The Ct of each sample was interpolated from the standard curves, and the number of miRNA copies was calculated by the iCycler iQ RealTime Detection System software (Bio-Rad Laboratories).

miRNA Transfection

Anti-miR™ miRNA Inhibitors (Ambion, Austin, Tex.) are chemically modified, single stranded nucleic acids designed to specifically bind and inhibit endogenous microRNA (miRNA) molecules. These ready-to-use inhibitors can be introduced into cells via a similar transfection strategy used for siRNAs, thereby facilitating the study of miRNA biological effects. Anti-miR™ miR-532-5p miRNA and Anti-miR™ negative control were transfected into a melanoma cell line using the reverse transfection protocol recommended by the manufacturer. In brief, siPORT NeoFX Transfection Agent (Ambion) was diluted in Opti-MEM medium (Invitrogen, Carlsbad, Calif.). Anti-miR™ miR-532-5p miRNA (Ambion) was also diluted in Opti-MEM medium for a final concentration of 30 nM. The diluted transfection reagent was combined with the diluted miRNA duplex followed by incubation at room temperature for 10 min. The mixture was dispensed into an empty 6 well dish and then seeded at 2.3×10⁵ cells per well. The same amount of negative control miRNA duplex was also transfected. Total RNA was extracted at 72 hr post-transfection and used for the mRNA qRT assay. Additional transfections were performed to analyze RUNX3 protein expression by flow cytometry.

### Flow Cytometry

Transfected cells (1×10⁶) were fixed and permeabilized by BD Cytofix/Cytoperm kit (BD Biosciences, San Jose, Calif.) and incubated at 4° C. for 1 hr with RUNX3 goat polyclonal Ab (1 μg) (Santa Cruz Biotechnology, Inc., Santa Cruz, Calif.) or an isotype matched control antibody. Rabbit anti-goat IgG-FITC (Santa Cruz Biotechnology, Inc.) was used as secondary antibody. Flow cytometry was performed using FACSCalibur (Becton Dickinson, Mountain View, Calif.) and data were analyzed with Cell Quest software (Becton Dickinson).

### Biostatistical Analysis

In primary and metastatic PE melanomas, comparisons of RUNX3/GAPDH mRNA expression in normal PE tissues were performed across all AJCC stages using the Kruskal-Wallis test. In primary melanomas, RUNX3 expression was correlated with age at diagnosis, Breslow thickness, and Clark level using Spearman's rank correlation; differences in RUNX3 expression according to AJCC stage, gender, Clark level, histologic subtype, and presence of ulceration were assessed via the Kruskal-Wallis test or Wilcoxon two-sample test as appropriate. The RUNX3 expression in metastatic melanoma tumors was correlated with patient age at diagnosis and gender in a univariate analysis.

A Cox regression model was used to identify predictors of 5-year overall survival. After finding that AJCC stage II and III primary tumors showed very similar survival curves, we combined these two groups. Factors included in the model were ulceration, Breslow depth (mm), AJCC stage, Clark level, gender and RUNX3 expression. Potential predictors of overall 5-year survival were entered into the multivariate model using a backward elimination method. Hazard ratios (HR) and 95% confidence intervals were reported for each variable.

RUNX3 mRNA and miR-532-5p expression in AJCC stages I, II, and III primary melanoma tumors were correlated using the Spearman rank correlation test.

### Results

mRNA Expression of RUNX3 in Melanoma Cell Lines

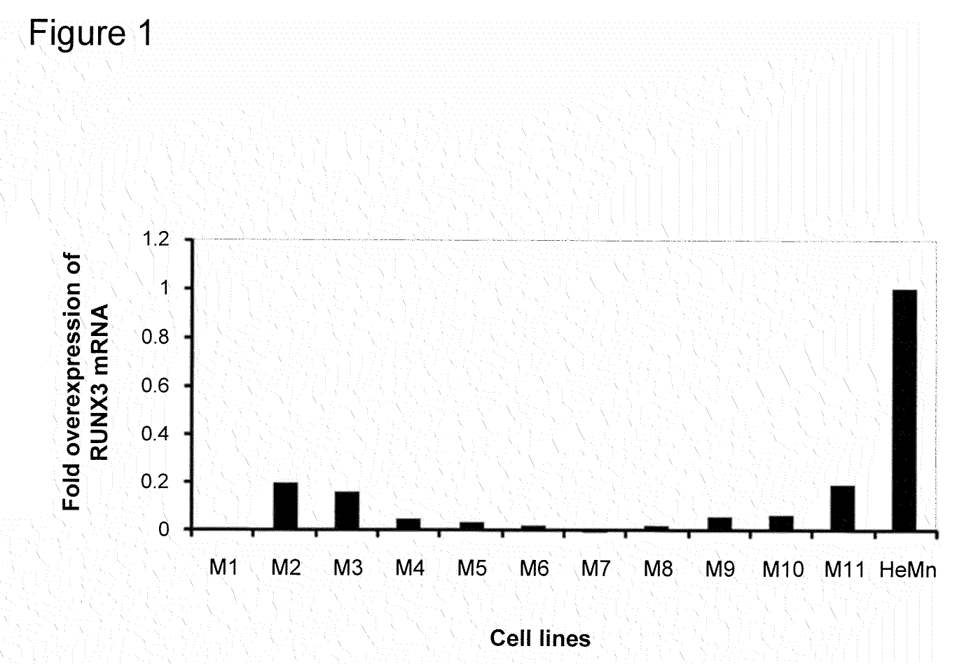

Initially, in studying alteration of RUNX3 expression in melanoma, expression of RUNX3 mRNA in 11 established melanoma lines and a normal human melanocyte line were assessed. Relative to normal melanocyte RUNX3 gene expression, all 11 established melanoma lines (FIG. 1) demonstrated significantly lower RUNX3 gene expression (p<0.001). On determining this finding, we went on to assess RUNX3 expression in primary and metastatic cutaneous melanomas.

### RUNX3 Expression in Primary and Metastatic Melanoma Tumors

There were 56 women and 67 men included in this study. The mean age of the study population was 65 years (median=67 yrs) and the median time of clinical follow-up for the study was 44 months (range, 3 to 149 mos). Patient and tumor characteristics studied are presented in Table 1. Briefly, 123 melanoma tumors from 123 patients were assayed in this study. Of these, 82 were primary tumors (AJCC stage I, N=45; AJCC stage II, N=21; AJCC stage III, N=16). The histopathology included superficial spreading (N=45, 54.9%), nodular (n=19, 23.1%), acral lentiginous (N=4, 4.9%), lentigo maligna (N=6, 7.3%), desmoplastic (N=8, 9.8%).

The mean RUNX3 mRNA expression was significantly different in comparison of normal skin versus AJCC stages I, II, and III primary melanomas (p=0.02). RUNX3 expression in AJCC stages I, II, and III primary melanomas was significantly lower than RUNX3 expression in normal skin samples (p=0.01, p=0.02, and p=0.01, respectively). RUNX3 expression demonstrated a nonlinear association with AJCC stage. No significant correlations between RUNX3 and known prognostic factors such as age, gender, Breslow thickness, Clark level, primary tumor ulceration, or histopathology were found.

Of the 123 melanomas assayed for this study, 41 were metastatic tumors (AJCC stage III, N=19; AJCC stage 1V, N=22). The mean RUNX3 mRNA expression was significantly down-regulated among melanoma metastases versus normal tissue overall (Kruskal-Wallis, p<0.0001). In comparison of AJCC stage 1V melanoma metastases to primary melanomas (AJCC stages I, II, III) RUNX3 mRNA expression was significantly (p=0.0004; Wilcoxon) downregulated. RUNX3 mRNA expression was also significantly downregulated in AJCC stage 1V melanoma metastases relative to normal tissues (p=0.0006). Decreased RUNX3 mRNA correlated with decreased RUNX3 protein expression, as was confirmed by flow cytometry analysis on melanoma cell lines using a specific anti-RUNX3 antibody.

### Survival Analysis

Overall survival was assessed regarding RUNX3 expression in primary cutaneous melanomas. In analysis of AJCC stages II/III primary melanoma patients (N=35), significant factors predicting overall survival in the multivariate model demonstrated that Clark level (HR 5.27, CI 1.35-20.56; p=0.02), gender (HR 4.38, CI 1.13-16.95; p=0.03), and RUNX3 mRNA expression (HR 1.01, CI 1.00-1.02; p=0.02) were significant. With these three variables included in the multivariate model, AJCC stage, ulceration, and Breslow depth did not significantly influence overall survival. The multivariate analysis demonstrated that RUNX3 downregulation expression in metastatic melanomas was related to disease outcome. We then investigated potential mechanisms for RUNX3 downregulation in metastatic melanoma cells.

### RUNX3 Promoter Region Hypermethylation

Because downregulation of RUNX3 mRNA expression has been related to gene promoter region CpG island hypermethylation in other cancers, we examined this epigenetic regulatory mechanism in cell lines, and primary and metastatic melanoma specimens. Aberrant promoter region hypermethylation of CpG islands is thought to play an important role in the inactivation of many tumor-suppressor genes in cancers. Specifically, hypermethylation of the RUNX3 promoter region has been shown to downregulate RUNX3 expression in other malignancies (8, 11). We assessed the promoter region hypermethylation of RUNX3 in melanoma by methylation-specific PCR analysis. Five of 17 (29%) melanoma lines assayed showed evidence of RUNX3 promoter region methylation. Of 82 melanoma specimens assessed, 7 (9%) demonstrated evidence of RUNX3 DNA hypermethylation. Only 2 of 52 (4%) primary melanomas demonstrated RUNX3 DNA hypermethylation, while 5 of 30 (16.7%) of metastatic melanomas demonstrated hypermethylation. The results demonstrated that promoter region hypermethylation is unlikely to play a significant role in the downregulation of RUNX3 expression during melanoma metastasis. However, the analysis demonstrated that hypermethylation of RUNX3 frequency increased only slightly in metastatic tumors.

miR-532-5p Expression in Melanoma

We next focused our attention on miRNA, another mechanism by which mRNA expression may be regulated (24, 25). Searching through the miRBase database (21), we found a specific miRNA sequence to RUNX3 mRNA. The miR-532-5p was a candidate miRNA to target the RUNX3 gene according to miRBase Targets version 3 (see the website microrna.sanger.ac.uk/targets/v3/). For miR-532-5p, the miRNA sequence is 5′-CAUGCCUUGAGUGUAGGACCGU-3′. The underlined sequences (ugCCAGGAUgUGAGUUCCGUAc) on miR-532-5p binds to RUNX3 mRNA (UAGGUCCUAGCAGAAGGCAUU). The miR-532-5p is complementary to the 3′ UTR sequence of the RUNX3 gene. We believed that miR-532-5p may be highly expressed in melanoma and suppresses RUNX3 mRNA expression.

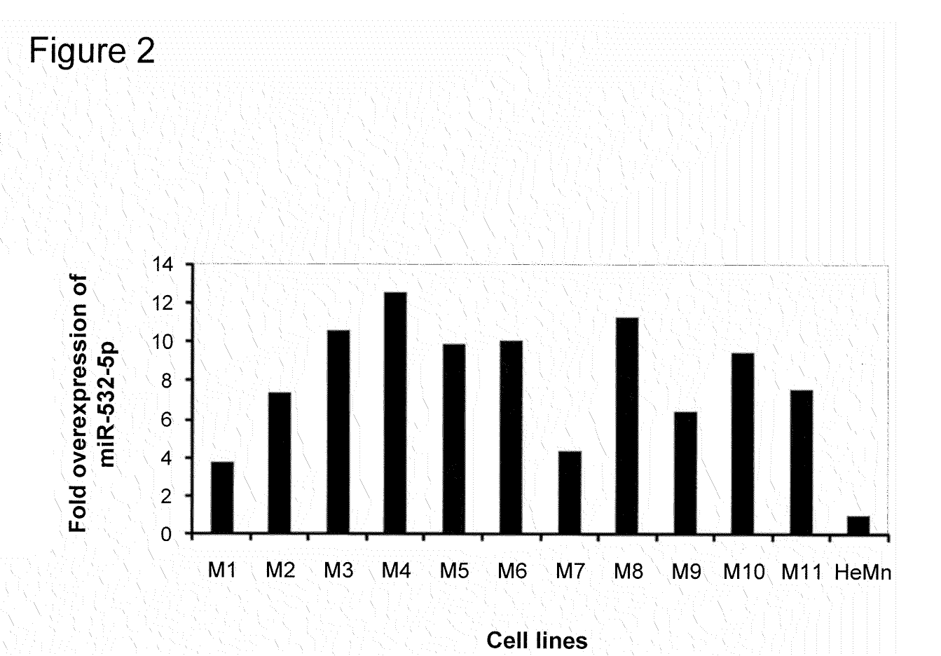

Eleven established cell lines and a normal melanocyte cell line were assessed for the expression of miR-532-5p. Higher miR-532-5p expression was seen in 11 of 11 established metastatic melanoma cell lines relative to normal melanocytes (FIG. 2).

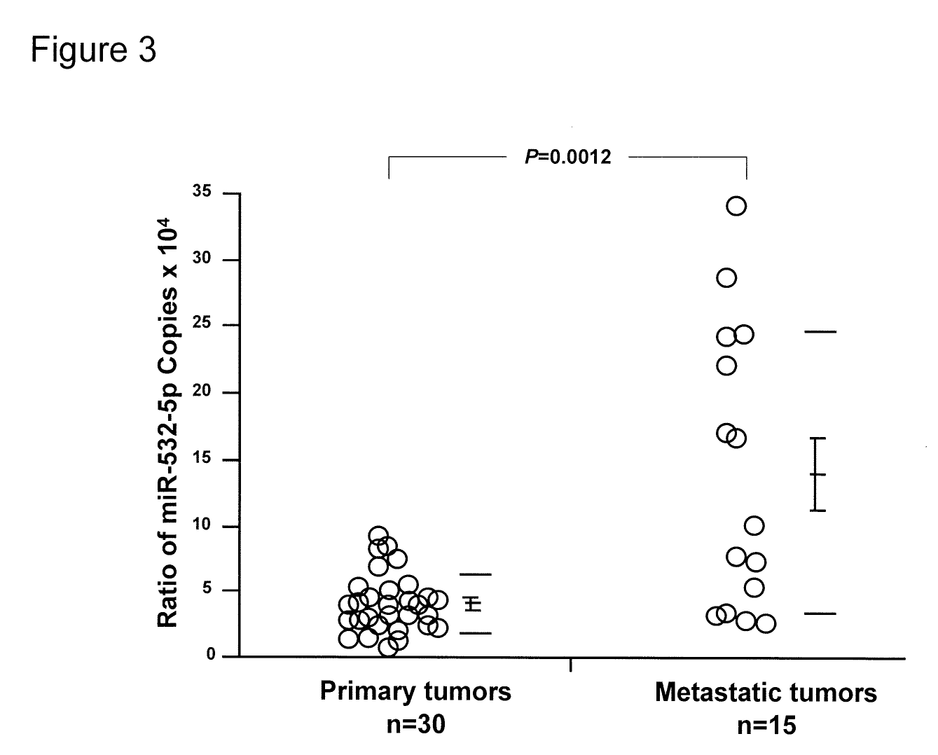

The miR-532-5p expression in PE metastatic melanoma tumors was analyzed and shown to be significantly higher than in primary melanomas (p=0.0012; FIG. 3). These results demonstrated that miR-532-5p was upregulated in melanoma as progression from primary to systemic metastasis occurs. There was an overall inverse relation of RUNX3 mRNA expression and miR-532-5p expression.

RUNX3 Activated by Anti miR-532 in Melanoma

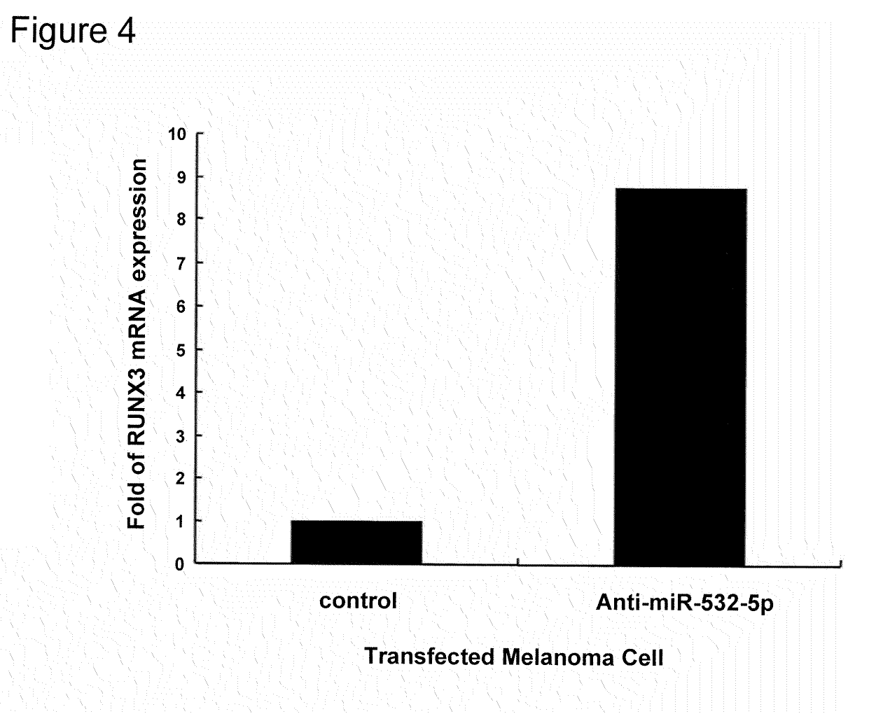

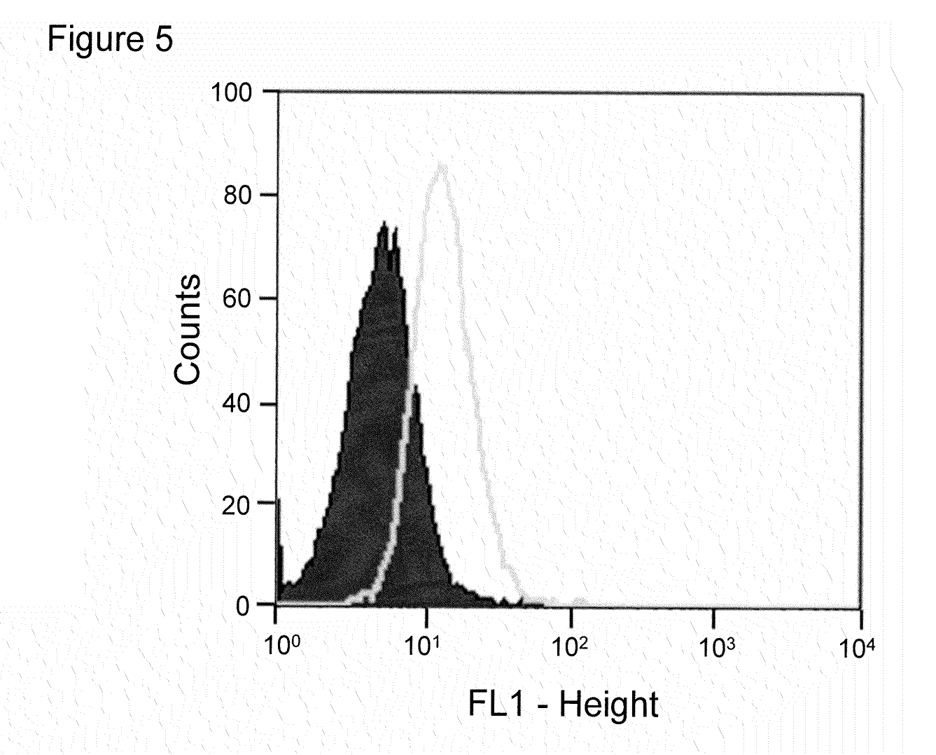

To validate that miR-532-5p inhibits the RUNX3 expression in melanoma, we transfected melanoma cells with anti-miR™ miR-532-5p miRNA (complementary sequences with miR-532-5p, Ambion) which was designed to inhibit miR-532-5p. RUNX3 mRNA expression in anti-miR-532-5p miRNA-transfected melanoma cells was up-regulated relative to anti-miR negative control-transfected melanoma cells (FIG. 4). RUNX3 protein expression was also upregulated in anti-miR-532 miRNA-transfected melanoma cells compared to non-transfected cells as demonstrated by flow cytometry (FIG. 5). These results demonstrated that inhibition of miR-532-5p up-regulated the RUNX3 expression in melanoma cells at the mRNA and protein level and indicated that miR-532-5p can inhibit the RUNX3 at the mRNA level.

### Discussion

Although present in many cell types, the role of RUNX3 in normal cellular development is not well understood. It is most prominent in the dorsal root ganglia, hematopoietic cells, and gastrointestinal tract, where it is thought to play a role in cell differentiation and development (2). In humans, loss of RUNX3 expression has been related to promoter region CpG island hypermethylation in several cancers (26-28), particularly in gastric cancer development and progression (2, 11). RUNX3 has been implicated as a tumor suppressor gene. RUNX3 has not been previously examined with respect to cutaneous melanoma; this is, to our knowledge, the first report describing abnormal RUNX3 expression in primary and metastatic cutaneous melanomas.

Our results demonstrated that RUNX3 mRNA expression was more suppressed in primary melanomas than in normal tissues, and further more suppressed in metastatic melanomas compared to normal tissues. This indicated a role for RUNX3 gene expression in melanoma development and progression. In general, RUNX3 expression in melanoma may play a similar important role as a tumor suppressor gene as in gastric cancer, but regulation is through a different mechanism (7, 11, 29). Interestingly, recent studies have shown that RUNX3 expression is relevant in developmental neurogenesis (30). RUNX3 is suggested to regulate neuron differentiation functions (31). Melanocytes from which cutaneous melanoma is derived have a neuroectodermal origin (32). Mueller et al. have also recently identified in glioblastomas that RUNX3 promoter region was hypermethylated in 56% of tumors (26).

Oddly, in melanoma, hypermethylation of the promoter region of RUNX3 does not play a major role in regulation as in other tumors (2, 11). Our results showed low frequency of hypermethylation of the RUNX3 promoter region in melanoma lines and melanoma tumors. These results suggested that RUNX3 expression in melanoma is likely suppressed by other epigenetic regulatory mechanisms other than hypermethylation. RUNX3 is located on chromosome 1p36, which previously has been shown to be a site of genomic deletions in cutaneous melanoma (33). Previously, we have shown that allelic imbalance of the microsatellite region of 1p36 in melanoma tumors can be up to 38%. However, these allele imbalances do not always interpret to loss of specific gene expression in that region. Nevertheless, this genomic region has been under considerable scrutiny for putative tumor-related genes.

Mature miRNAs are 19 to 25 nucleotide noncoding RNA molecules that can down-regulate various gene products by translational repression (25, 34). This occurs when partially complementary sequences are present in the 3′ untranslated regions (3′UTR) of the target mRNAs or by directing mRNA degradation (25). miRNAs can be expressed in a tissue-specific manner and are considered to play important roles in cell proliferation, apoptosis, and differentiation during mammalian development (24, 34, 35). Moreover, recent studies have shown a link between patterns of miRNA expression and the development of cancer (36) and downregulation of specific cancer-related genes (37-39). miR-532-5p, which had a complementary sequence to the 3′ UTR region, was assessed as a candidate miRNA targeting the RUNX3 mRNA as a potential down-regulating mechanism. We believed that miR-532-5p is highly expressed in melanoma and may suppress RUNX3 expression. The results demonstrated that miR-532-5p expression is significantly increased in melanoma cell lines and metastatic melanoma compared with normal melanocytes and primary melanomas, respectively. Moreover, we demonstrated that inhibition of miR-532-5p upregulated RUNX3 mRNA and protein expression in melanoma lines. These findings demonstrated that miR-532-5p regulates RUNX3 expression in melanomas. The studies also suggest that miR-532-5p may play a role as a regulatory factor in melanoma progression. The miRNA-532-5p is located on chromosome region Xp11.23, whereby there is several other miR located nearby in that region.

In melanoma patients, RUNX3 mRNA expression was a significant predictor of overall survival. Although the influence of RUNX3 expression on survival was dominated by more significant factors such as Clark level and gender, it remained a more significant predictor of survival than Breslow depth, AJCC stage, and tumor ulceration in the small sample size assessed.

Melanoma metastasis is commonly associated with a poor prognosis, and therefore targeting these mechanisms may lead to more effective treatments for patients. Therapeutic strategies to decrease miR-532-5p may potentially be useful for limiting melanoma metastasis. Further work is warranted to evaluate the role of miR-532-5p and to develop therapeutic strategies targeting miR-532-5p in vivo. Moreover, aberrantly expressed miRNA, such as miR-532-5p, may be a useful biomarker for diagnosis and prognosis in melanoma. Recent advances in techniques for the identification of miRNA should facilitate the use of clinical specimens for this purpose. The identification of critical targets for individual RUNX3 miRNAs may provide novel insights into the mechanisms of progression in melanoma.

We have shown in this study that RUNX3 can be suppressed by both miR and hypermethylation of the promoter region. Previously, we have shown that the 1p36 region where RUNX3 is located has allelic imbalance. These three types of molecular aberrations collectively may suppress RUNX3 during development and metastasis of melanoma. The role of RUNX3 in melanoma progression is not known but may follow similar mechanistic pathways as found in of other cancers. A recent study has found that RUNX3 forms a ternary complex with β-catenin/TCF4 and attenuates Wnt signaling (40). Wnt signaling is known to play an important role in melanoma progression (41).

### REFERENCES

1. Balch C M, Soong S J, Atkins M B, et al. An evidence-based staging system for cutaneous melanoma. CA Cancer J Clin 2004; 54:131-49.
2. Ito Y. Oncogenic potential of the RUNX gene family: ‘overview’. Oncogene 2004; 23:4198-208.
3. Araki K, Osaki M, Nagahama Y, et al. Expression of RUNX3 protein in human lung adenocarcinoma: implications for tumor progression and prognosis. Cancer Sci 2005; 96:227-31.
4. Cohen M M, Jr. RUNX genes, neoplasia, and cleidocranial dysplasia. Am J Med Genet 2001; 104:185-8.
5. Hiramatsu T, Osaki M, Ito Y, Tanji Y, Tokuyasu N, Ito H. Expression of RUNX3 protein in human esophageal mucosa and squamous cell carcinoma. Pathobiology 2005; 72:316-24.
6. Li J, Kleeff J, Guweidhi A, et al. RUNX3 expression in primary and metastatic pancreatic cancer. J Clin Pathol 2004; 57:294-9.
7. Oshimo Y, Oue N, Mitani Y, et al. Frequent loss of RUNX3 expression by promoter hypermethylation in gastric carcinoma. Pathobiology 2004; 71:137-43.
8. Wei D, Gong W, Oh S C, et al. Loss of RUNX3 expression significantly affects the clinical outcome of gastric cancer patients and its restoration causes drastic suppression of tumor growth and metastasis. Cancer Res 2005; 65:4809-16.
9. Javed A, Barnes G L, Pratap J, et al. Impaired intranuclear trafficking of Runx2 (AML3/CBFA1) transcription factors in breast cancer cells inhibits osteolysis in vivo. Proc Natl Acad Sci USA 2005; 102:1454-9.
10. Young D W, Hassan M Q, Pratap J, et al. Mitotic occupancy and lineage-specific transcriptional control of rRNA genes by Runx2. Nature 2007; 445:442-6.
11. Li Q L, Ito K, Sakakura C, et al. Causal relationship between the loss of RUNX3 expression and gastric cancer. Cell 2002; 109:113-24.
12. Hussein M R, Roggero E, Tuthill R J, Wood G S, Sudilovsky O. Identification of novel deletion Loci at 1p36 and 9p22-21 in melanocytic dysplastic nevi and cutaneous malignant melanomas. Arch Dermatol 2003; 139:816-7.
13. Poetsch M, Dittberner T, Woenckhaus C. Microsatellite analysis at 1p36.3 in malignant melanoma of the skin: fine mapping in search of a possible tumour suppressor gene region. Melanoma Res 2003; 13:29-33.
14. Hoon D S, Spugnardi M, Kuo C, Huang S K, Morton D L, Taback B. Profiling epigenetic inactivation of tumor suppressor genes in tumors and plasma from cutaneous melanoma patients. Oncogene 2004; 23:4014-22.
15. Takeuchi H, Fujimoto A, Tanaka M, Yamano T, Hsuch E, Hoon D S. CCL21 chemokine regulates chemokine receptor CCR7 bearing malignant melanoma cells. Clin Cancer Res 2004; 10:2351-8.
16. Koyanagi K, Kuo C, Nakagawa T, et al. Multimarker quantitative real-time PCR detection of circulating melanoma cells in peripheral blood: relation to disease stage in melanoma patients. Clin Chem 2005; 51:981-8.
17. Koyanagi K, O'Day S J, Gonzalez R, et al. Serial monitoring of circulating melanoma cells during neoadjuvant biochemotherapy for stage III melanoma: outcome prediction in a multicenter trial. J Clin Oncol 2005; 23:8057-64.
18. Spugnardi M, Tommasi S, Dammann R, Pfeifer G P, Hoon D S. Epigenetic inactivation of RAS association domain family protein 1 (RASSF1A) in malignant cutaneous melanoma. Cancer Res 2003; 63:1639-43.
19. Umetani N, de Maat M F, Mori T, Takeuchi H, Hoon D S. Synthesis of universal unmethylated control DNA by nested whole genome amplification with phi29 DNA polymerase. Biochem Biophys Res Commun 2005; 329:219-23.
20. Umetani N, Takeuchi H, Fujimoto A, Shinozaki M, Bilchik A J, Hoon D S. Epigenetic inactivation of ID4 in colorectal carcinomas correlates with poor differentiation and unfavorable prognosis. Clin Cancer Res 2004; 10:7475-83.
21. Griffiths-Jones S, Grocock R J, van Dongen S, Bateman A, Enright A J. miRBase: microRNA sequences, targets and gene nomenclature. Nucleic Acids Res 2006; 34:D140-4.
22. Chen C, Ridzon D A, Broomer A J, et al. Real-time quantification of microRNAs by stem-loop RT-PCR. Nucleic Acids Res 2005; 33:e179.
23. Tang F, Hajkova P, Barton S C, Lao K, Surani M A. MicroRNA expression profiling of single whole embryonic stem cells. Nucleic Acids Res 2006; 34:e9.
24. Bartel D P. MicroRNAs: genomics, biogenesis, mechanism, and function. Cell 2004; 116:281-97.
25. He L, Hannon G J. MicroRNAs: small RNAs with a big role in gene regulation. Nat Rev Genet 2004; 5:522-31.
26. Mueller W, Nutt C L, Ehrich M, et al. Downregulation of RUNX3 and TES by hypermethylation in glioblastoma. Oncogene 2007; 26:583-93.
27. Nomoto S, Kinoshita T, Mori T, et al. Adverse prognosis of epigenetic inactivation in RUNX3 gene at 1p36 in human pancreatic cancer. Br J Cancer 2008; 98:1690-5.
28. Sakakura C, Miyagawa K, Fukuda K I, et al. Frequent silencing of RUNX3 in esophageal squamous cell carcinomas is associated with radioresistance and poor prognosis. Oncogene 2007; 26:5927-38.
29. Kim T Y, Lee H J, Hwang K S, et al. Methylation of RUNX3 in various types of human cancers and premalignant stages of gastric carcinoma. Lab Invest 2004; 84:479-84.
30. Inoue K, Shiga T, Ito Y. Runx transcription factors in neuronal development. Neural Develop 2008; 3:20.
31. Nakamura S, Senzaki K, Yoshikawa M, et al. Dynamic regulation of the expression of neurotrophin receptors by Runx3. Development 2008; 135:1703-11.
32. Silver D, Pavan W The origin and development of neural crest-derived melanocytes. In: VJ H and SP L, editors. From Melanocytes to Melanoma. Totowa: Humana Press; 2006. p. 3-26.
33. Fujiwara Y, Chi D D, Wang H, et al. Plasma DNA microsatellites as tumor-specific markers and indicators of tumor progression in melanoma patients. Cancer Res 1999; 59:1567-71.
34. Ambros V. The functions of animal microRNAs. Nature 2004; 431:350-5.
35. Sempere L F, Freemantle S, Pitha-Rowe I, Moss E, Dmitrovsky E, Ambros V. Expression profiling of mammalian microRNAs uncovers a subset of brain-expressed microRNAs with possible roles in murine and human neuronal differentiation. Genome Biol 2004; 5:R13.
36. Meltzer P S. Cancer genomics: small RNAs with big impacts. Nature 2005; 435:745-6.
37. Bemis L T, Chen R, Amato C M, et al. MicroRNA-137 targets microphthalmia-associated transcription factor in melanoma cell lines. Cancer Res 2008; 68:1362-8.
38. Calin G A and Croce C M. MicroRNA signatures in human cancers. Nat Rev Cancer 2006; 6:857-66.
39. Zhang B, Pan X, Cobb G P, Anderson T A. microRNAs as oncogenes and tumor suppressors. Dev Biol 2007; 302:1-12.
40. Ito K, Lim A C, Salto-Tellez M, et al. RUNX3 attenuates beta-catenin/T cell factors in intestinal tumorigenesis. Cancer Cell 2008; 14:226-37.
41. Lin Y C, You L, Xu Z, et al. Wnt inhibitory factor-1 gene transfer inhibits melanoma cell growth. Hum Gene Ther 2007; 18:379-86.

All publications cited herein are incorporated by reference in their entirety.

What is claimed is:

1. A method of detecting melanoma, comprising:
providing a test biological sample from a subject; and
determining the RUNX3 (Runt-related transcription factor 3) gene expression or protein activity level in the test sample,
wherein the RUNX3 gene expression or protein activity level in the test sample, if lower than that in a normal sample, indicates that the subject is likely to be suffering from melanoma.

2. The method of claim 1, wherein the melanoma is primary or metastatic.

3. The method of claim 1, wherein the RUNX3 gene expression level is determined at the mRNA or protein level.

4. A method of detecting melanoma, comprising:
providing a first sample containing melanoma cells; and
determining the RUNX3 gene expression or protein activity level in the first sample,
wherein the RUNX3 gene expression or protein activity level in the first sample, if lower than that in a second sample containing melanoma cells, indicates that the melanoma in the first sample is likely to be at a more advanced stage than that in the second sample.

5. The method of claim 4, wherein the RUNX3 gene expression level is determined at the mRNA or protein level.

6. A method of predicting the outcome of melanoma, comprising:
providing a first sample containing melanoma cells from a first subject; and
determining the RUNX3 gene expression or protein activity level in the first sample,
wherein the RUNX3 gene expression or protein activity level in the first sample, if higher than that in a second sample containing melanoma cells from a second subject, indicates that the overall survival of the first subject is likely to be longer than that of the second subject.

7. The method of claim 6, wherein the RUNX3 gene expression or protein activity level is determined at the mRNA or protein level.

8. A method of detecting cancer, comprising:
providing a test biological sample from a subject; and
determining the expression level of miR-532-5p in the test sample,
wherein the expression level of miR-532-5p in the test sample, if higher than that in a normal sample, indicates that the subject is likely to be suffering from cancer.

9. The method of claim 8, wherein the cancer is melanoma, breast cancer, gastric cancer, pancreas cancer, colon cancer, or esophagus cancer.

10. The method of claim 8, wherein the cancer is primary or metastatic.

11. The method of claim 8, wherein the RUNX3 gene expression or protein activity level in the test sample is lower than that in the normal sample.

12. A method of detecting cancer, comprising:
providing a first sample containing cancer cells; and
determining the expression level of miR-532-5p in the first sample,
wherein the expression level of miR-532-5p in the first sample, if higher than that in a second sample containing cancer cells, indicates that the cancer in the first sample is likely to be at a more advanced stage than that in the second sample.

13. The method of claim 12, wherein the cancer is melanoma, breast cancer, gastric cancer, pancreas cancer, colon cancer, or esophagus cancer.

14. The method of claim 12, wherein the cancer is primary or metastatic.

15. The method of claim 12, wherein the RUNX3 gene expression or protein activity level in the first sample is lower than that in the second sample.

16. A method of reducing the inhibition of RUNX3 by miR-532-5p, comprising:
providing a cell expressing a RUNX3 gene and an miR-532-5p gene; and
contacting the cell with an agent that interferes with the interaction between RUNX3 and miR-532-5p transcripts.

17. The method of claim 16, wherein the cell is a cancer cell.

18. The method of claim 17, wherein the cancer is melanoma, breast cancer, gastric cancer, pancreas cancer, colon cancer, or esophagus cancer.

19. The method of claim 16, wherein the agent is an anti-miR-532-5p miRNA.
